(19) United States
(12) Patent Application Publication
(10) Pub. No.: US 2021/0353330 A1
(43) Pub. Date: Nov. 18, 2021

(54) ROBOTIC UTERINE MANIPULATORS

(71) Applicant: Covidien LP, Mansfield, MA (US)

(72) Inventors: Scott J. Prior, Branford, CT (US); Arvind Rajagopalan Mohan, Dracut, MA (US); Nikolai D. Begg, Wellesley, MA (US); Kevin R. Slisz, Old Saybrook, CT (US); Zachary Traina, Verona, NJ (US)

(21) Appl. No.: 17/318,493

(22) Filed: May 12, 2021

Related U.S. Application Data
(60) Provisional application No. 63/023,376, filed on May 12, 2020.

(51) Int. Cl. A61B 17/42; A61B 17/34; A61B 34/30

(52) U.S. Cl. CPC A61B 17/4241; A61B 17/3478; A61B 2017/4216; A61B 2034/303; A61B 34/30

(57) ABSTRACT

A uterine manipulator includes an elongated shaft assembly, a colpotomy cup supported on the elongated shaft assembly, and a tip assembly. The tip assembly extends distally from the colpotomy cup and has a piercing tip configured to pierce tissue.

### CROSS REFERENCE TO RELATED APPLICATIONS

This application claims the benefit of U.S. Provisional Application Ser. No. 63/023,376, filed May 12, 2020, the entire contents of which are incorporated by reference herein.

### TECHNICAL FIELD

This disclosure relates to hysterectomy and, more particularly, to robotic uterine manipulators.

### BACKGROUND

Colpotomy, one of the final steps in a hysterectomy, requires making a circular incision in vaginal tissue to separate the uterus from the vagina with a cutting tool such as an electrosurgical instrument. This incision is typically performed with the aid of a uterine manipulator. Uterine manipulators are conventionally used during laparoscopic hysterectomy procedures to position the vagina and the cervix to facilitate separation and to enable removal of the uterus or other tissue specimens subsequent to performance of a colpotomy.

### SUMMARY

In accordance with an aspect of this disclosure, a uterine manipulator includes an elongated shaft assembly, a colpotomy cup supported on the elongated shaft assembly and a tip assembly. The tip assembly extends distally from the colpotomy cup and has a piercing tip configured to pierce tissue.

In aspects, the piercing tip may be spring loaded by a spring. The tip assembly may include an elongated tube that supports the piercing tip. The spring may be supported in the elongated tube. The piercing tip may be positioned to move relative to the elongated tube from a retracted position within the elongated tube to an extended position distally beyond the elongated tube. The spring may be operatively coupled to a drive mechanism of the uterine manipulator. The drive mechanism may be actuatable to cause the spring to expand. Expansion of the spring may cause the piercing tip to move from the retracted position to the extended position.

In accordance with another aspect of this disclosure, a uterine manipulator system includes an anchor configured to secure to a fundus, a grasper for grasping the anchor to control a position of the fundus, and a uterine manipulator. The uterine manipulator includes an elongated shaft assembly, a colpotomy cup supported on the elongated shaft assembly, and a tip assembly extending distally from the colpotomy cup and having a piercing tip configured to pierce the fundus.

In accordance with still another aspect of this disclosure, a robotic uterine manipulator system includes a robotic arm and a uterine manipulator coupled to the robotic arm. The uterine manipulator includes an elongated shaft assembly, a colpotomy cup supported on the elongated shaft assembly, and a tip assembly extending distally from the colpotomy cup and having a piercing tip configured to pierce a fundus.

Other aspects, features, and advantages will be apparent from the description, the drawings, and the claims that follow.

### BRIEF DESCRIPTION OF THE DRAWINGS

The accompanying drawings, which are incorporated in and constitute a part of this specification, illustrate aspects of this disclosure and, together with a general description of this disclosure given above, and the detailed description given below, serve to explain the principles of this disclosure, wherein:

FIG. 1 is a perspective view of a robotic system in accordance with the principles of this disclosure;

FIG. 2 is an enlarged, perspective view of a proximal portion of a robotic uterine manipulator of the robotic colpotomy system of FIG. 1;

FIG. 3 is an enlarged view of a distal portion of the robotic uterine manipulator inserted transvaginally into a patient's uterus with a tip assembly thereof shown in a first position;

FIG. 4 is an enlarged view of FIG. 3 with the tip assembly of the robotic uterine manipulator shown in a second position; and

FIG. 5 is an enlarged view of the distal portion of the robotic uterine manipulator inserted transvaginally into a patient's uterus with an anchor secured to a fundus of the patient and a grasper shown grasping the anchor.

### DETAILED DESCRIPTION

Aspects of this disclosure are described in detail with reference to the drawings, in which like reference numerals designate identical or corresponding elements in each of the several views. As used herein, the term “distal” refers to that portion of structure farther from the user, while the term “proximal” refers to that portion of structure, closer to the user. As used herein, the term “clinician” refers to a doctor, nurse, or other care provider and may include support personnel.

In the following description, well-known functions or constructions are not described in detail to avoid obscuring the present disclosure in unnecessary detail.

Robotic surgical systems have been used in minimally invasive medical procedures and can include robotic arm assemblies. Such procedures may be referred to as what is commonly referred to as “Telesurgery.” Some robotic arm assemblies include one or more robot arms to which surgical instruments can be coupled. Such surgical instruments include, for example, electrosurgical forceps, cutting instruments, staplers, graspers, electrocautery devices, or any other endoscopic or open surgical devices. Prior to or during use of the robotic surgical system, various surgical instruments can be selected and connected to the robot arms for selectively actuating end effectors of the connected surgical instruments. Some of these surgical instruments utilize electrical energy, for example, to effectuate electrocautery.

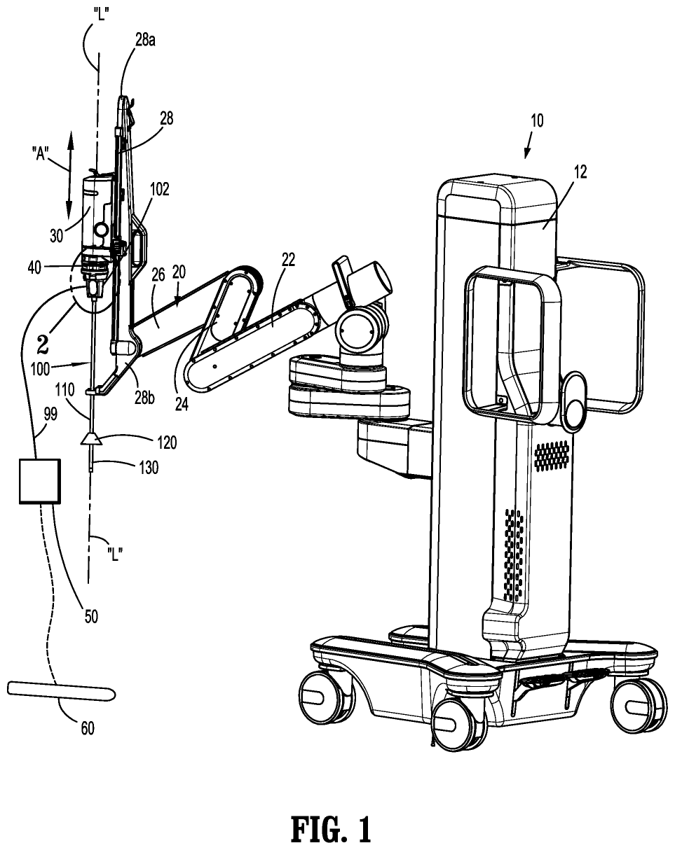

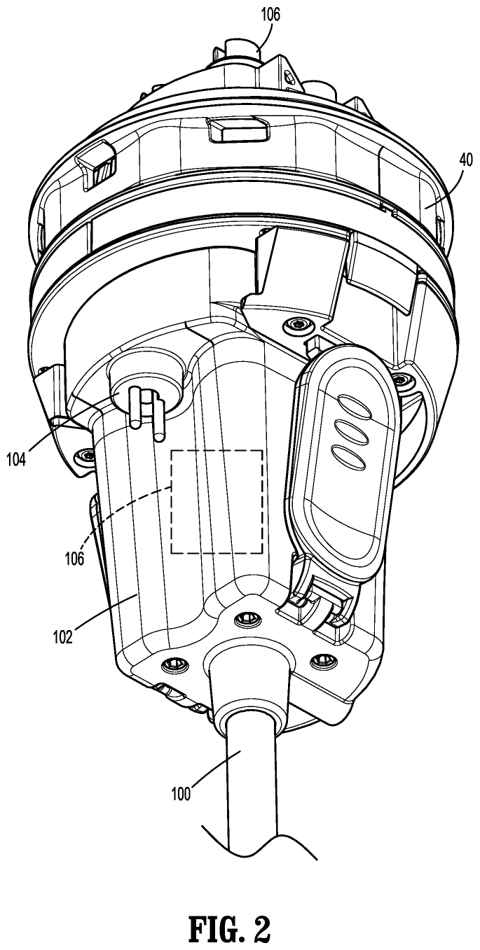

With reference to FIGS. 1 and 2, a robotic surgical system, such as the robotic colpotomy system 10 illustrated in FIG. 1, includes a robotic arm assembly 20 that supports a surgical instrument, such as a uterine manipulator 100, for effectuating a surgical procedure (e.g., a colpotomy), an instrument drive unit 30 that imparts driving force to uterine manipulator 100, and a sterile interface module 40 that enables a proximal housing assembly 102 of uterine manipulator 100 to interface with instrument drive unit 30. This interface advantageously maintains sterility, provides a means to transmit electrical communication between robotic colpotomy system 10 and uterine manipulator 100, provides a means for transferring torque (e.g., rotational force) from robotic colpotomy system 10 (e.g., IDU 30) to uterine manipulator 100 for performing a function (e.g., sealing, cutting, manipulating, etc.) with uterine manipulator 100 and/or provides a means to selectively attach/remove uterine manipulator 100 to robotic colpotomy system 10 (e.g., for rapid instrument exchange). For a more detailed description of similar sterile interface modules and components thereof, reference can be made to WO2017205308 by Zemlock et al., the entire contents of which are incorporated by reference herein.

Robotic colpotomy system 10 further includes an energy source such as an electrosurgical generator 50 that couples to uterine manipulator 100 and/or any number of other surgical instruments such as an electrosurgical probe or an electrocautery blade 60 via an electrosurgical cable 99 and a connector assembly 104 supported by sterile interface module 40 and/or proximal housing assembly 102 of uterine manipulator 100. For a more detailed description of one example of an electrosurgical generator, reference can be made to U.S. Pat. No. 8,784,410, the entire contents of which are incorporated by reference herein. For a more detailed description of one example of connector assembly 104, reference can be made to U.S. Patent Application No. 62/823,036, filed Mar. 25, 2019, and entitled “Robotic Surgical Systems with Electrical Switch of Instrument Attachment,” the entire contents of which are incorporated by reference herein. For a more detailed description of one example of an electrocautery blade 60, reference can be made to U.S. Pat. No. 8,128,622 or 8,460,289, the entire contents of each of which are incorporated herein by reference.

Robotic colpotomy system 10 employs various robotic elements to assist the clinician and allow remote operation (or partial remote operation) of surgical instrumentation such as uterine manipulator 100. Various robotic arms, gears, cams, pulleys, electric and mechanical motors, etc. may be employed for this purpose and may be designed with robotic colpotomy system 10 to assist the clinician during the course of an operation or treatment, and which can be included with, and/or part of one or more drive mechanisms 106 of uterine manipulator 100, sterile interface module 40, and/or instrument drive unit 30. Such robotic systems may include remotely steerable systems, automatically flexible surgical systems, remotely flexible surgical systems, remotely articulating surgical systems, wireless surgical systems, modular or selectively configurable remotely operated surgical systems, etc.

Robotic colpotomy system 10 includes a medical work station (not shown) that may be employed with one or more consoles positioned next to the operating theater or located in a remote location. In this instance, one team of clinicians may prep the patient for surgery and configure robotic colpotomy system 10 with uterine manipulator 100 while another clinician (or group of clinicians) remotely controls uterine manipulator 100 via the one or more consoles. As can be appreciated, a highly skilled clinician may perform multiple operations in multiple locations without leaving his/her remote console. This can be economically advantageous and a benefit to the patient or a series of patients. For a detailed description of exemplary medical work stations and/or components thereof, reference may be made to U.S. Pat. No. 8,828,023 and PCT Application Publication No. WO2016/025132, the entire contents of each of which are incorporated by reference herein.

With continued reference to FIG. 1, robotic arm assembly 20 of robotic surgical system 10 includes a cart 12 having robotic arms 22, 24, 26 that are pivotally coupled together and movable together and/or relative to one another and cart 12. Robotic arm 26 is coupled to a slide rail 28 that supports instrument drive unit (“IDU”) 30 and sterile interface module 40 for operating uterine manipulator 100. IDU 30 defines a longitudinal axis “L” and is slidably supported on slide rail 28 and selectively axially movable along longitudinal axis “L,” as indicated by arrows “A,” between a proximal position adjacent a proximal end portion 28a of slide rail 28, and a distal position adjacent a distal end portion 28b of slide rail 28.

Robotic surgical system 10 can be in the form of an electrosurgical colpotomy system. In general, components of the electrosurgical colpotomy system can be used to effectuate a colpotomy. Briefly, when using a uterine manipulator for colpotomy during a laparoscopic hysterectomy, a colpotomy cup can be used as a backstop for a clinician to circumferentially cut along with a laparoscopic tool (e.g., radiofrequency or “RF” tool) around the uterus. To make such a circumferential cut uniform, the clinician is required to determine the location of a rim of the colpotomy cup. Indeed, to identify the exact location of the rim, the clinician may be required to repeatedly move the uterine manipulator as the cut is made. This movement can be cumbersome and/or time consuming, particularly when clinician must also coagulate and transect uterine arteries in order to minimize blood loss during the colpotomy.

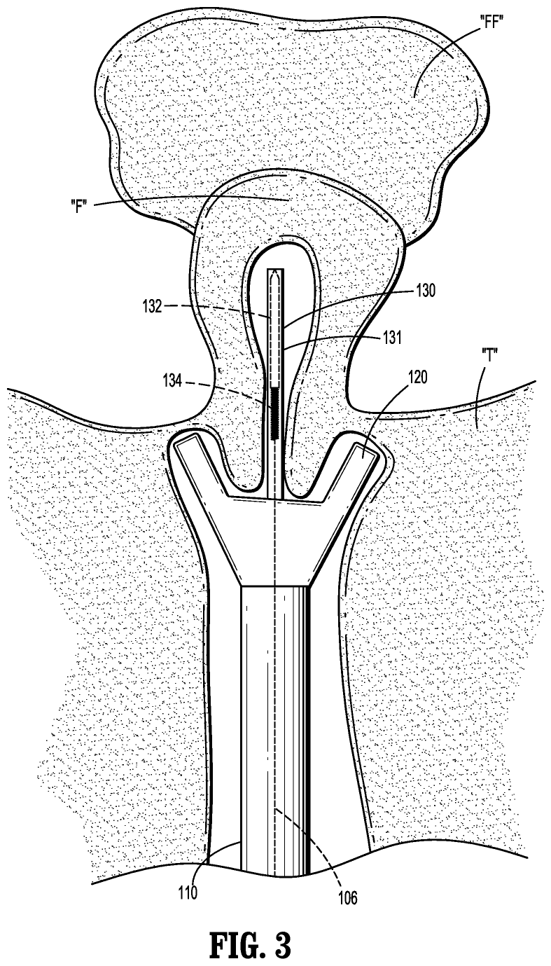

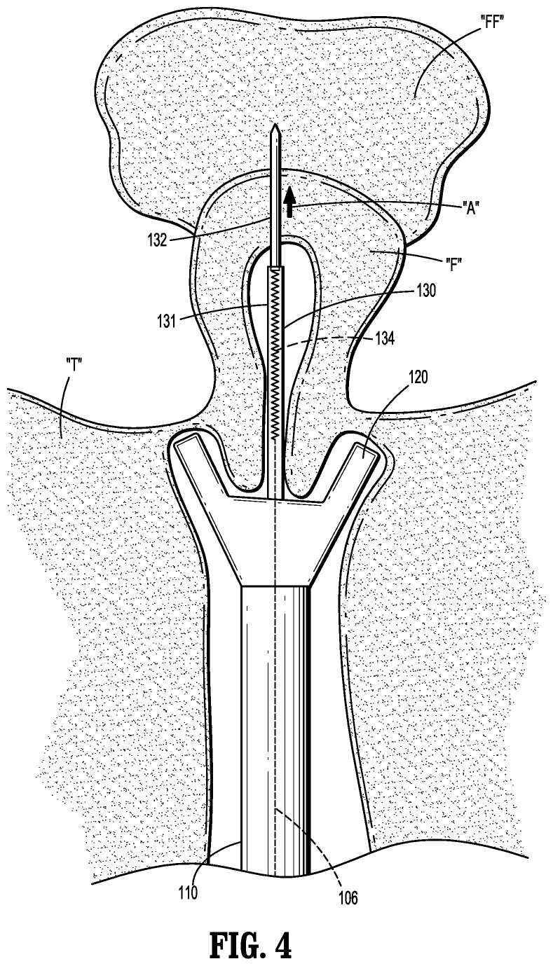

With reference to FIGS. 1, 3, and 4, in order to improve force and control of tissue for effectuating, for example, a colpotomy procedure, uterine manipulator 100 includes an elongated shaft assembly 110 that extends from proximal housing assembly 102 and supports a colpotomy cup 120 and a tip assembly 130. Tip assembly 130 includes an elongated tube 131 that extends distally from colpotomy cup 120 and moveably supports a piercing tip 132 that is spring-loaded by a spring 134 (e.g., a compression spring, although any suitable spring may be utilized). Piercing tip 132 may have a pointed end and/or one or more sharpened edges (e.g., a bayonet style tip) for enabling piercing tip 132 to puncture tissue “T” such as fundus “F” and/or fundal fibroid “FF.” In response to actuation of one or more drive mechanisms 106 of uterine manipulator 100, piercing tip 132 is movable from a retracted position within elongated tube 131 (FIG. 3) to an extended position (FIG. 4) distally beyond elongated tube 131, as indicated by arrow “A,” for selectively piercing into and/or through fundus “F” and/or fundal fibroid “FF” to provide manipulation leverage for uterine manipulator 100.

In aspects, although described herein in connection with spring-loaded structure, piercing tip 132 can be actuated via any suitable electrical and/or mechanical structure for moving piercing tip 132 relative to elongated tube 131 (e.g., threaded rotation, cable actuation, magnetics, etc.).

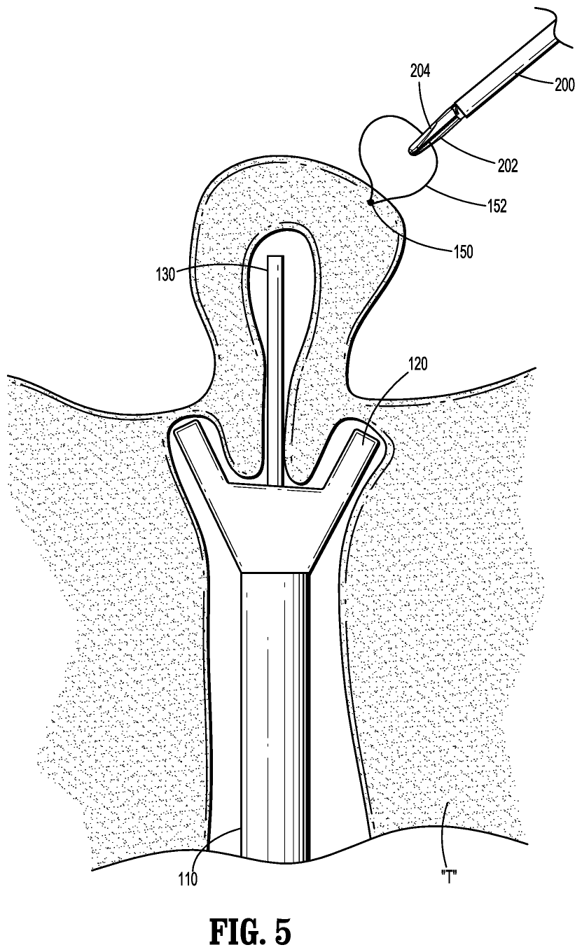

Certain anatomy may be difficult to manipulate solely with a uterine manipulator. Thus, as seen in FIG. 5, to further facilitate manipulation of tissue “T” such as the fundus “F,” an anchor 150 (e.g., a tack, screw, etc.) can be secured to fundus “F” (such as via a tack applier—not shown) so that a separate surgical instrument, such as a grasper 200 (with movable jaw members 202, 204), can grasp anchor 150 for further controlling and/or manipulating tissue “T.” Anchor 150 may include a tether or loop 152 to facilitate grasping. Grasper 200 may be a robotic and/or a hand-held grasper. For a more detailed description of exemplary grasping devices and/or tack appliers, reference can be made to U.S. Pat. Nos. 5,403,342, 10,258,359, and U.S. Patent Application Publication No. 2018/0214144, the entire contents of each of which are incorporated by reference herein.

Further, although detailed herein with respect to a robotic system, the disclosed uterine manipulators can be provided as manual and/or hand-held instruments. For a more detailed description of an exemplary hand-held uterine manipulator, reference can be made to U.S. Patent Application Publication No. 2018/0325554, the entire contents of which are incorporated by reference herein.

Securement of any of the components of the disclosed devices may be effectuated using known securement techniques such welding, crimping, gluing, fastening, etc.

Persons skilled in the art will understand that the structures and methods specifically described herein and shown in the accompanying figures are non-limiting exemplary aspects, and that the description, disclosure, and figures should be construed merely as exemplary of particular aspects. It is to be understood, therefore, that this disclosure is not limited to the precise aspects described, and that various other changes and modifications may be effectuated by one skilled in the art without departing from the scope or spirit of the disclosure. Additionally, the elements and features shown or described in connection with certain aspects may be combined with the elements and features of certain other aspects without departing from the scope of this disclosure, and that such modifications and variations are also included within the scope of this disclosure. Accordingly, the subject matter of this disclosure is not limited by what has been particularly shown and described.

What is claimed is:

1. A uterine manipulator comprising:
an elongated shaft assembly;
a colpotomy cup supported on the elongated shaft assembly; and
a tip assembly extending distally from the colpotomy cup and having a piercing tip configured to pierce tissue.

2. The uterine manipulator according to claim 1, wherein the piercing tip is spring loaded by a spring.

3. The uterine manipulator according to claim 2, wherein the tip assembly includes an elongated tube that supports the piercing tip.

4. The uterine manipulator according to claim 3, wherein the spring is supported in the elongated tube.

5. The uterine manipulator according to claim 4, wherein the piercing tip is positioned to move relative to the elongated tube from a retracted position within the elongated tube to an extended position distally beyond the elongated tube.

6. The uterine manipulator according to claim 5, wherein the spring is operatively coupled to a drive mechanism of the uterine manipulator, the drive mechanism actuatable to cause the spring to expand.

7. The uterine manipulator according to claim 6, wherein expansion of the spring causes the piercing tip to move from the retracted position to the extended position.

8. A uterine manipulator system comprising:
an anchor configured to secure to a fundus;
a grasper for grasping the anchor to control a position of the fundus; and
a uterine manipulator comprising:
an elongated shaft assembly;
a colpotomy cup supported on the elongated shaft assembly; and
a tip assembly extending distally from the colpotomy cup and having a piercing tip configured to pierce the fundus.

9. The uterine manipulator system according to claim 8, wherein the piercing tip is spring loaded by a spring.

10. The uterine manipulator system according to claim 9, wherein the tip assembly includes an elongated tube that supports the piercing tip.

11. The uterine manipulator system according to claim 10, wherein the spring is supported in the elongated tube.

12. The uterine manipulator system according to claim 11, wherein the piercing tip is positioned to move relative to the elongated tube from a retracted position within the elongated tube to an extended position distally beyond the elongated tube.

13. The uterine manipulator system according to claim 12, wherein the spring is operatively coupled to a drive mechanism of the uterine manipulator, the drive mechanism actuatable to cause the spring to expand.

14. The uterine manipulator system according to claim 13, wherein expansion of the spring causes the piercing tip to move from the retracted position to the extended position.

15. A robotic uterine manipulator system comprising:
a robotic arm; and
a uterine manipulator coupled to the robotic arm, the uterine manipulator comprising:
an elongated shaft assembly;
a colpotomy cup supported on the elongated shaft assembly; and
a tip assembly extending distally from the colpotomy cup and having a piercing tip configured to pierce a fundus.

16. The robotic uterine manipulator system according to claim 15, wherein the piercing tip is spring loaded by a spring.

17. The robotic uterine manipulator system according to claim 16, wherein the tip assembly includes an elongated tube that supports the piercing tip.

18. The robotic uterine manipulator system according to claim 17, wherein the spring is supported in the elongated tube.

19. The robotic uterine manipulator system according to claim 18, wherein the piercing tip is positioned to move relative to the elongated tube from a retracted position within the elongated tube to an extended position distally beyond the elongated tube.

20. The robotic uterine manipulator system according to claim 19, wherein the spring is operatively coupled to a drive mechanism of the uterine manipulator, the drive mechanism actuatable to cause the spring to expand and the piercing tip to move from the retracted position to the extended position.
